(19) United States
(12) Patent Application Publication
(10) Pub. No.: US 2026/0162780 A1
(43) Pub. Date: Jun. 11, 2026

(54) AN INTELLIGENT COMPUTER AIDED DECISION SUPPORT SYSTEM

(71) Applicant: CORTI APS, Copenhagen K (DK)

(72) Inventor: Lars Maaløe, Copenhagen K (DK)

(73) Assignee: CORTI APS, Copenhagen K (DK)

(21) Appl. No.: 18/698,858

(22) PCT Filed: Oct. 6, 2022
(86) PCT No.: PCT/EP2022/077826

§ 371 Date: Apr. 5, 2024

(30) Foreign Application Priority Data
Oct. 6, 2021 (DK) 202100957

(51) Int. Cl. G16H 10/20; G10L 25/66

(52) U.S. Cl. CPC G16H 10/20; G10L 25/66

(57) ABSTRACT

A method for recognizing information such as speech or text comprises providing a processing unit, and a memory including a database having an encoder model comprising a statistically learned model. The method further comprises inputting said information into the processing unit and processing the information including a recognizing routine comprising the encoder model. At a first time point, the encoder model is defined by a first observable variable and an encoder hierarchy of a first set of random variables. The encoder hierarchy is constituted by layers including a first layer with a first random variable, and a second layer with a second random variable. At a second time point, the encoder model is defined by a second observable variable and an encoder hierarchy constituting layers of a second set of random variables depending on the first set of random variables. The processing includes passing said information through said encoder model, and outputting recognized information.

The present specification relates to a method of assisting an interviewing party by a computer such that the interviewing party may decide a response action faster or more reliable. A statistically learned model is contemplated for assisting the interviewing party.

The setting can be an emergency situation such as a car accident, a cardiac arrest or an air plane experiencing problems.

An example of an intelligent computer aided decision support system is disclosed in WO20239910, which is incorporated in the present disclosure by reference.

A first aspect of the present disclosure is:

A method for recognizing information such as speech or text, comprising:
providing a processing unit, and a memory including a database having an encoder model comprising a statistically learned model,
inputting said information into said processing unit as an electronic signal, and processing said electronic signal,
said processing including a recognizing routine comprising said encoder model,
at a first time point said encoder model defined by a first observable variable and an encoder hierarchy of a first set of random variables,
said encoder hierarchy constituted by layers including a first layer with a first random variable, and a second layer with a second random variable,
at a second time point said encoder model defined by a second observable variable and an encoder hierarchy of a second set of random variables,
said encoder hierarchy constituted by layers including said first layer with a third random variable, and said second layer with a fourth random variable,
said third random variable dependent on said first random variable, and said fourth random variable dependent on said second random variable,
said processing including passing said information through said encoder model, and outputting recognized information.

A second aspect of the present disclosure is:

A method for generating information such as an image or speech, comprising:
providing a processing unit, and a memory including a database having a decoder model comprising a statistically learned model,
said decoder model defined by an observable variable and a decoder hierarchy of random variables,
said decoder hierarchy constituted by layers including a first layer and a second layer,
at a first time point said decoder model defined by a first observable variable and a decoder hierarchy of a first set of random variables,
said decoder hierarchy constituted by layers including a first layer with a first random variable, and a second layer with a second random variable,
at a second time point said decoder model defined by a second observable variable and a decoder hierarchy of a second set of random variables,
said decoder hierarchy constituted by layers including said first layer with a third random variable, and said second layer with a fourth random variable,
said third random variable dependent on said first random variable, and said fourth random variable dependent on said second random variable,
said method comprising sampling a value of the random variable of the top layer, and processing said value through said hierarchy such that said information being generated

A third aspect of the present disclosure is:

A method for learning a list of digits, for tasks such as natural language understanding tasks, detecting anomalies, and generating sound, as a compressed representation of an interview between an interviewing party and an interviewee party, said method comprising:

training or learning said encoder model of claim 1 and said decoder model of claim 2 by:
i) said encoder processing information and saving the distribution of each random variable from the encoder layers,
ii) said decoder processing the distributions from the encoder layers, and saving the distributions of each random variable of the decoder layers,
iii) determining a difference between a saved distribution from the encoder model and a saved distribution from the decoder model,
iv) minimizing the difference by varying parameters of said encoder model and said decoder model.

In the following specific examples according to aspects of the present disclosure will be explained in more detail with reference to the accompanying drawings. The present disclosure may, however, be embodied in different forms than depicted below, and should not be construed as limited to any examples set forth herein. Rather, any examples are provided so that the disclosure will be thorough and complete, and will fully convey the scope of the invention to those skilled in the art. Like reference numerals refer to like elements throughout. Like elements will, thus, not be described in detail with respect to the description of each figure.

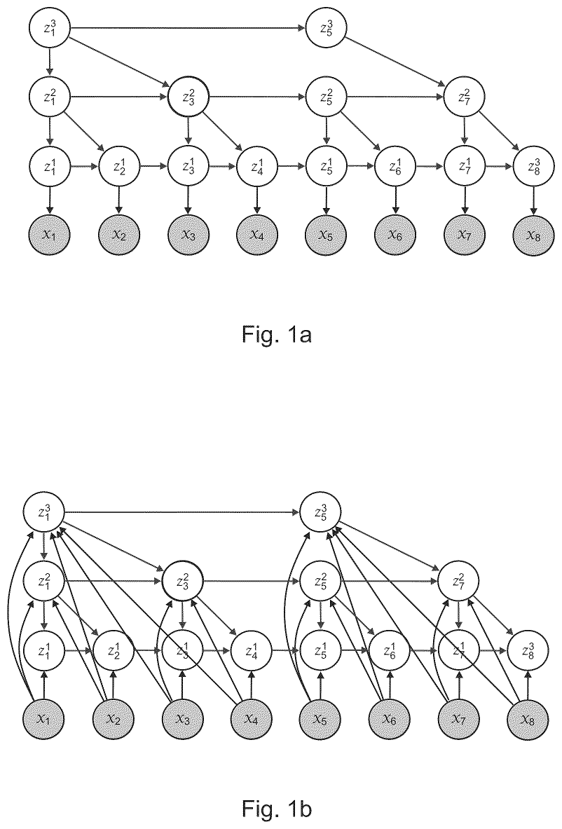

FIGS. 1a and 1b illustrate a decoder/“generative” model p(x,z) and an encoder/“inference model” q (z|x) respectively.

Three layers are shown, which may be a preferred setup, however two layers may be contemplated as well as more than three layers such as four layers or five layers.

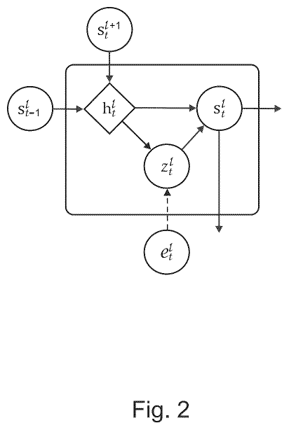

Blue arrows indicate parameter sharing between the inference and generative models. The deterministic variable is omitted in FIGS. 1a and 1b, but is shown in FIG. 2.

FIG. 2 shows the graphical model of the recurrent cell of the model in FIGS. 1a and 1b for a single time step, i.e. for

$${s}_{t}^{l}$$

uptake. All blue allows ale shared between generation and inference. The dashed arrow is used only during inference. The solid arrow has unique transformations during inference and generation. h is the deterministic variable.

The latent variable at timestep t and layer l∈[1, L] is denoted

$${z}_{t}^{l} .$$

Each latent layer is updated only every s timesteps, where sᵢ is a layer-dependent integer, or stride, defined in a way such that sᵢ is largest for larger values of l. This imposes the inductive bias that latent variables exist at different temporal resolutions with z^{l} changing over longer time scales than z^{l-1}.

In speech, phonetic variation (10-400 ms), morphological and semantic features at the word level and speaker-related variation at the global scale make this a reasonable assumption.

The timesteps at which a layer updates its latent state are given by T defined by:

$$\{ t ∈ [ 1 , T ] \vert t mod {s}_{l} = 1 \}$$

In practice and equivalently, this is represented by having references to the unique states copied over time:

$${z}_{t}^{l} ≡ {z}_{m {ax}_{𝒯} \{ τ ∈ {𝒯}_{l} \vert τ \leq t \}}^{l}$$

The joint distribution factorizes over time and over the latent hierarchy:

$$p ( x , z ) = {\underbrace{( {\prod }_{t} p ( {x}_{t} \vert {z}_{t}^{l} ) )}}_{reconstruction terms} {\underbrace{( {\prod }_{l = 1}^{L} {\prod }_{t ∈ {𝒯}_{l}} p ( {z}_{t}^{l} \vert {z}_{t - 1}^{l} , {z}_{t}^{l + 1} ) )}}_{state transitions}$$

The inference model similarly factorizes over time and over the layers of the latent hierarchy with the posterior conditioned on a span of the observed variable x_{l:t÷s}_{ₗ} dependent on the layer stride sₗ:

$$q ( z \vert x ) = {\prod }_{l = 1}^{L} q ( {z}^{l} \vert x ) = {\prod }_{l = 1}^{L} {\prod }_{t ∈ {𝒯}_{l}} q ( {z}_{t}^{l} \vert {z}_{t - 1}^{l} , {z}_{t}^{l + 1} , {x}_{t : t + {s}_{l}} )$$

An encoder

$${φ}_{ewvae}^{enc , l}$$

is used to parameterize the approximate posterior q(z|x) which is taken to be an isotropic Gaussian. This encoder may be the same for all stochastic layers l, be layer-specific or use a ladder-network.

In the speech domain the observed variable can have sample rates of tens of thousands of frames per second. This results in sequence lengths that are not generally feasible to model with recurrent architectures. For this reason, we chose s₁ to be much greater than 1 such as greater than 20 or 40 or 60 to achieve an initial temporal downsampling and set sₗ=c^{j-1}sₗ for l>1 and some constant c. For instance setting s₁=64, c=8 results in s₂=512.

The encoder may be designed as a ladder-network as this provides some benefits compared to alternatives. Specifically, a ladder-network leverages parameter sharing across the latent hierarchy and importantly processes the full observed sequence only once and shares the resulting representations for all latent variables. This yields a more computationally efficient encoder and a higher activity in latent variables towards the top of the hierarchy.

The encoder/decoder networks are parametrized using 1D convolutions that operate on the raw waveform.

As shown in the zoomed in illustration of FIG. 2,

$${s}_{t}^{l}$$

is split into stochastic

$${z}_{t}^{l}$$

and deterministic hₜ^{l} parts. The deterministic state is computed using the top-down and temporal context, which then conditions the stochastic state at that level. The stochastic variables follow diagonal Gaussians with predicted means and variances.

The choice of output distribution [symbol] (ρ_{x,t}) is generally data dependent and at the most fundamental level it may be selected either as continuous or discrete distributions depending on the type of x, the likelihoods of which are not comparable. ρ_{x,t} is a set of parameters.

x denotes a waveform sampled with some bit-depth, e.g., b=16 which entails that x∈{0, 1, . . . , 2^{b}-1} and is discrete-valued. x may be scaled to take values between −1 and 1.

x becomes approximately continuous as b becomes large.

Nonetheless, continuous distributions, and especially mixtures of continuous distributions, may yield arbitrarily high likelihoods when used to model discrete data.

To correctly model discrete x with a continuous distribution x must be dequantized to be continuous by for example adding uniform noise or using a variational approach. The continuous likelihood obtained via dequantization has been shown to be a lower bound on the likelihood that could have been obtained with a discrete distribution.

The dataset for training may be a speech dataset, for example containing 16 kHz recordings of 630 speakers of eight major dialects of American English, each reading ten phonetically rich sentences. This amounts to 6300 total recordings splits approximately in 3.94 hours of audio for training and 1.43 hours of audio.

What is claimed is:

1. A method for recognizing information such as speech or text, comprising:
providing a processing unit, and a memory including a database having an encoder model comprising a statistically learned model,
inputting said information into said processing unit as an electronic signal, and processing said electronic signal,
said processing including a recognizing routine comprising said encoder model,
at a first time point said encoder model defined by a first observable variable and an encoder hierarchy of a first set of random variables,
said encoder hierarchy constituted by layers including a first layer with a first random variable, and a second layer with a second random variable,
at a second time point said encoder model defined by a second observable variable and an encoder hierarchy of a second set of random variables,
said encoder hierarchy constituted by layers including said first layer with a third random variable, and said second layer with a fourth random variable,
said third random variable dependent on said first random variable, and said fourth random variable dependent on said second random variable,
said processing including passing said information through said encoder model, and outputting recognized information.

2. A method for generating information such as an image or speech, comprising:
providing a processing unit, and a memory including a database having a decoder model comprising a statistically learned model,
said decoder model defined by an observable variable and a decoder hierarchy of random variables,
said decoder hierarchy constituted by layers including a first layer and a second layer,
at a first time point said decoder model defined by a first observable variable and a decoder hierarchy of a first set of random variables,
said decoder hierarchy constituted by layers including a first layer with a first random variable, and a second layer with a second random variable,
at a second time point said decoder model defined by a second observable variable and a decoder hierarchy of a second set of random variables,
said decoder hierarchy constituted by layers including said first layer with a third random variable, and said second layer with a fourth random variable,
said third random variable dependent on said first random variable, and said fourth random variable dependent on said second random variable,
said method comprising sampling a value of the random variable of the top layer, and processing said value through said hierarchy such that said information being generated.

3. A method for learning a list of digits, for tasks such as natural language understanding tasks, detecting anomalies, and generating sound, as a compressed representation of an interview between an interviewing party and an interviewee party, said method comprising:
training or learning said encoder model of claim 1 by:
i) said encoder processing information and saving the distribution of each random variable from the encoder layers,
ii) determining a difference between a saved distribution from the encoder model and a saved distribution from a decoder model, and
iii) minimizing the difference by varying parameters of said encoder model and said decoder model.

4. The method according to claim 1, providing a sound recorder and capturing the sound of an interviewee party during an interview between an interviewing party and said interviewee party.

5. The method according to claim 1, said encoder model receiving as input a respective number of samples of the sound of said interviewee party.

6. The method according to claim 1, said information being cardiac arrest or an acute disease such as meningitis, presented during an interview between an interviewing party and an interviewee party.

7. The method according to claim 1, said first observable variable constitute said information.

8. The method according to claim 1, each encoder layer is dependent on another encoder layer, except for the first, which is dependent on the observable variable.

9. The method according to claim 1, each random variable of said encoder having a probability distribution.

10. The method according to claim 2, each random variable of said decoder represent a probability distribution.

11. The method according to claim 2, wherein each decoder layer is dependent on another an encoder layer, except for the first, which is dependent on the observable variable.

12. The method according to claim 1, said first layer being lower in said encoder hierarchy than said second layer.

13. The method according to claim 1, said first set of random variables being jointly distributed according to a prior probability distribution.

14. The method according to claim 1, said second set of random variables being jointly distributed according to a prior probability distribution.

15. A method for learning a list of digits, for tasks such as natural language understanding tasks, detecting anomalies, and generating sound, as a compressed representation of an interview between an interviewing party and an interviewee party, said method comprising:
training or learning said decoder model of claim 2 by:
i) said decoder processing distributions from encoder layers, and saving the distributions of each random variable of the decoder layers,
ii) determining a difference between a saved distribution from an encoder model and a saved distribution from the decoder model,
iii) minimizing the difference by varying parameters of said encoder model and said decoder model.
